(19) United States
(12) Patent Application Publication
(10) Pub. No.: US 2010/0047353 A1
(43) Pub. Date: Feb. 25, 2010

(54) HAIR CARE COMPOSITION

(76) Inventors: Ashish Batra, Lake Jeckson, TX (US); Susan L. Jordan, Doylestown, PA (US); Xiaodong Zhang, Livingston, NJ (US); Michael Meerbote, Gutenberg (DE); Klaus Schiller, Halle (DE); Elke Nieter, Holleben (DE)

Correspondence Address: The Dow Chemical Company, Intellectual Property Section, P.O. Box 1967, Midland, MI, 48641-1967, US

(21) Appl. No.: 12/532,216

(22) PCT Filed: Mar. 20, 2008
(86) PCT No.: PCT/US08/57586

§ 371 Date: Sep. 21, 2009

Related U.S. Application Data
(60) Provisional application No. 60/919,210, filed on Mar. 21, 2007.

(51) Int. Cl. A61K 9/14; A61K 8/72; A61Q 5/00

(52) U.S. Cl. 424/489; 424/70.11; 510/119

(57) ABSTRACT

A hair care composition which comprises a plurality of cross-linked polymer particles, said polymer being the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid is useful for controlling sebum on hair without providing undue whiteness to the hair.

### FIELD OF THE INVENTION

The present invention relates to an aqueous hair care compositions, particularly to hair conditioners or shampoos.

### BACKGROUND OF THE INVENTION

Human hair becomes soiled due to its contact with the surrounding environment and from the sebum secreted by the scalp.

European Patent No. 678 294 discloses an aqueous composition for cleansing human hair which comprises a) 1-20 weight percent of at least one anionic, non-ionic amphoteric or zwitterionic surfactant and b) 0.25-7.5 weight percent of at least one water-insoluble, particle-shaped, synthetic, cross-linked copolymer comprising a C₁-C₁₈-alkyl methacrylate, at least another monomer having more than one polymerizable double bond, and optionally further unsaturated polymerizable compounds; and c) 0.25-7.5 weight percent of at least one compound selected from the group of hydroxy-C₁₋₄-alkyl cellulose(s), carboxymethyl and carboxyethyl celluloses and the water-soluble salts thereof, and/or optionally neutralized polymers containing carboxylic groups. The aqueous composition in the examples of the European Patent comprises an ethanediol methacrylate/lauryl methacrylate copolymer which is commercially available as Polytrap Q5-6603. The aqueous composition is applied to dry hair, left on the hair for 10-15 minutes and removed by washing the hair with a conventional shampoo. The composition delays re-fatting of the hair and reduces the frequency at which the hair has to be washed. Unfortunately, these compositions may leave an undesirable degree of whiteness on the hair and require application in the dry state followed by rinsing. This is both inconvenient, requiring additional time and effort, and requires use of a product form separate from the routines of washing and conditioning hair.

WO 03/028678 discloses hair treatment compositions comprising 5 to 50 weight percent of a detersive surfactant and at least about 0.025 weight percent hollow particles having a mean particle size of less than about 80 microns. The publication discusses that scalp secretions (i.e. sebum and other oily material) tend to travel along hair fibers and cause a collapse of hair fibers. This results in hair looking oily and dirty. The publication discloses that the hollow particles deposited on the hair surface act as spacing units in-between the fibers and prevent collapse of the hair fiber onto each other. The resulting maintenance of hair fibers separation is said to allow hair to look and feel cleaner for a longer period of time than the hair washed with a non-particle containing composition. Such hollow particles are commercially available, for example, from Kobo Products Inc. under the designation Silica Shells. Unfortunately, leave-on hair conditioners comprising these hollow particles leave an undesirable degree of whiteness on hair.

Allyl methacrylate polymers are sold under the trademark Polypore E-200 by Amcol Health & Beauty Solutions Inc. Arlington Heights, Ill. (USA). These polymers are advertised as a means for stabilizing and protecting fragrances, moisturizers and other sensitive ingredients in high pH/harsh environments of hair colors and relaxers. The oil sorption properties of such polymers are disclosed in U.S. Pat. No. 5,834,577. Unfortunately, also the disclosed allyl methacrylate polymers leave an undesirable degree of whiteness on hair.

There is a need to find hair care compositions which are useful for sebum control on hair without leaving an undesirable degree of whiteness on hair.

### SUMMARY OF THE INVENTION

One aspect of the present invention is an aqueous hair care composition comprising a plurality of cross-linked polymer particles, said polymer being the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid.

Another aspect of the present invention is a method of providing an aqueous hair care composition with sebum-retaining properties, comprising the step of incorporating a plurality of cross-linked polymer particles in the hair care composition, said polymer being the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid.

Yet another aspect of the present invention is the use of a plurality of cross-linked polymer particles, said polymer being the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid as a sebum-controlling active in a hair care composition.

Yet another aspect of the present invention is a method of cleansing human hair which comprises the steps of applying the above-mentioned hair care composition to the hair and subsequently rinsing the hair care composition off the hair.

Yet another aspect of the present invention is a method of conditioning human hair which comprises the steps of applying the above-mentioned hair care composition to the hair and drying the hair or letting the hair dry without removing the hair care composition.

Yet another aspect of the present invention is the use of the above-mentioned hair care composition for cleansing or conditioning human hair.

### SHORT DESCRIPTION OF THE DRAWINGS

FIG. 1 illustrates the sebum control of a hair care composition of the present invention and of a control composition for various amounts of sebum applied.

FIG. 2 illustrates the sebum control of a control hair care composition over time.

FIG. 3 illustrates the sebum control of a hair care composition of the present invention over time.

FIG. 4 is another illustration of the sebum control of a hair care composition of the present invention and of a control hair care composition over time.

FIG. 5 illustrates hair tresses treated with a hair care composition of the present invention and with known hair care compositions.

### DETAILED DESCRIPTION OF THE INVENTION

It has surprisingly been found that an aqueous hair care composition described in more detail hereafter is useful for sebum control on hair without leaving an undesirable degree of whiteness on hair. The hair care composition is useful for imbibing sebum thus suppressing oily shine on hair.

The aqueous hair care composition is preferably in the form of a shampoo or in the form of a leave-on or rinse-off hair conditioner.

The aqueous hair care composition comprises a plurality of cross-linked polymer particles. The polymer is the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid. The cross-linked polymer particles and methods of preparing them are described in U.S. Pat. Nos. 4,489,058 and 4,619,826. These patents disclose the use of the cross-linked polymers for controlling acne. The polymers are able to imbibe and retain sebum. The International Publications WO 92/00719 and WO 92/00724 disclose a makeup composition and a cosmetic lotion comprising the above-mentioned cross-linked polymer for imbibing sebum. However, none of these prior art documents discloses the benefit of the cross-linked polymer particles in aqueous hair care compositions, specifically its use for sebum control on hair without leaving an undesirable degree of whiteness on hair. To the contrary, WO 92/00724 discloses that a microsuspension of the sebum-imbibing cross-linked polymers in water would form a whitish film when applied to the user's face. WO 92/00724 teaches the use of a naturalizing agent to reduce light scattering by the sebum-imbibing polymer and to form a smooth film of substantially uniform refractive index to reduce the whitish appearance of the film when applied to the user's face. Polypropylene is recommended as a naturalizing agent.

The cross-linked polymer is the polymerization product of at least two monomer units selected from the group consisting of monoalkenyl aromatic compounds, alkyl esters derived from a saturated alcohol and acrylic or methacrylic acid, and vinyl esters of an aliphatic carboxylic acid. Preferably the cross-linked polymer is the polymerization product of two of the above-mentioned monomer units, the amount of each of the monomer units being from 25 to 75 weight percent, more preferably from 30 to 70 weight percent, based on the total weight of the monomer units. In addition to these monomer units, the cross-linked polymer generally comprises a minor amount of the cross-linking agent as described further below. The cross-linking agent can be any di- or poly-functional compound. The cross-linked polymer does not comprise a monomer unit with more than one polymerizable double bond other than the cross-linking agent.

The preferred monoalkenyl aromatic compounds which may be utilized in the preparation of the polymers for the aqueous hair care composition of the present invention contain a straight or branched chain monoalkenyl residue of from 2 to about 10 carbon atoms and may optionally be ring substituted with halogen or a straight or branched chain alkyl moiety of from 1 to about 20 carbon atoms, more preferably from 1 to about 12 carbon atoms. Such compounds include, for example, various halostyrenes such as 2-chlorostyrene, 3-fluorostyrene, 4-fluorostyrene and the like; vinyl naphthalenes, allylbenzene, 2-phenyl-2-butene, styrene and various substituted styrenes such as alkylstyrenes. Such alkylstyrenes include, for example, n-alkylstyrenes such as methylstyrene (i.e., vinyl toluene), n-butylstyrene, n-amylstyrene, n-octylstyrene, or n-octadecylstyrene; isoalkylstyrenes such as isobutylstyrene, isohexylstyrene, or isododecylstyrene; sec-alkylstyrenes such as sec-butylstyrene, sec-hexylstyrene, or sec-octylstyrene; tertiary-alkylstyrenes such as tert-butylstyrene, tert-amylstyrene, 3,5-ditert-butylstyrene, 4-tert-hexylstyrene, tert-octylstyrene, or tert-eicosylstyrene. The most preferred monoalkenyl aromatic compounds are styrene and a styrene ring substituted with a straight or branched chain alkyl moiety of from 1 to about 12 carbon atoms.

The preferred alkyl esters derived from a saturated alcohol and acrylic or methacrylic acid which may be utilized in the preparation of the polymers for the aqueous hair care composition of the present invention are acrylate or methacrylate esters derived from an alcohol moiety containing from 1 to about 20, preferably 8 to 20, carbon atoms. Such esters include, for example, butyl methacrylate, butyl acrylate, hexyl acrylate, isobornyl methacrylate, lauryl methacrylate, cetyl methacrylate, eicosyl acrylate, the mixed ester cetyl-eicosyl methacrylate, lauryl methacrylate, stearyl methacrylate, isobornyl acrylate, and lauryl acrylate.

The preferred vinyl esters of aliphatic carboxylic acids used in the preparation of the polymers for the aqueous hair care composition of the present invention are esters prepared from carboxylic acids containing 2 to about 20, preferably 8 to 20, carbon atoms such as vinyl acetate, vinyl butyrate, vinyl stearate, or vinyl 2-ethylhexoate.

The particularly preferred polymers are crosslinked polymers of styrene and lauryl methacrylate; vinyl toluene and lauryl methacrylate; polymers of tertiary-butylstyrene with lauryl methacrylate, stearyl methacrylate or vinyl stearate; terpolymers of tertiary-butylstyrene, 2-ethylhexyl acrylate and lauryl methacrylate; terpolymers of tertiary-butylstyrene, 2-ethylhexyl acrylate and stearyl methacrylate; polymers of isobornyl methacrylate and lauryl methacrylate; and polymers of vinyl stearate and lauryl methacrylate or isobornyl methacrylate.

More preferably, the aqueous hair care composition of the present invention comprises a plurality of cross-linked polymer particles wherein the polymer is the polymerization product of two alkyl esters derived from a saturated alcohol and acrylic or methacrylic acid.

Most preferably, the cross-linked polymer particles are cross-linked copolymers of isobornyl methacrylate and lauryl methacrylate. The copolymer is preferably made from 30 to 75, more preferably from 40 to 70, weight percent of isobornylmethacrylate and from 70 to 25, more preferably from 60 to 30 weight percent of laurylmethacrylate, based on the total weight of isobornyl methacrylate and lauryl methacrylate.

The cross-linked polymer particles in general comprise from about 0.01 to about 5 weight percent, preferably about 0.1 to about 2 weight percent, more preferably about 0.3 to about 1 weight percent of cross-linking agent, based on total weight of the polymer. The cross-linking agent can be any di- or poly-functional compound known to be useful as a cross-linking agent such as divinylbenzene, diethylene glycol dimethacrylate, diisopropenylbenzene, diallyl maleate, diallyl phthalate, allyl acrylates, allyl methacrylates, allyl fumarates, allyl itaconates, cyclooctadiene, divinyl phthalates, vinyl isopropenyl benzene, or other di or polyethylenically unsaturated cross-linking agents described, for example, in U.S. Pat. No. 3,520,806.

The particle size diameter of the cross-linked polymer utilized in the aqueous hair care composition of the present invention may vary, but in general, the particles have a volume average particle size of from about 0.5 to about 500 micrometers, more preferably about 2 to about 100 micrometers, most preferably about 2 to about 30 micrometers at their smallest diameters. The volume average particle size is measured with a Malvern Mastersizer 2000 light scattering analyzer.

Cross-linked polymer particles with a volume average particle size of from about 2 to 30 micrometers at their smallest diameter and an average aspect ratio of from 1 to 2, preferably from 1 to 1.5, more preferably from 1 to 1.2, are particularly preferred since they are generally invisible to the naked eye and do not scatter light to a high degree in hair care compositions. Such cross-linked polymer particles minimize the whitening effect on hair. The average aspect ratio as defined herein is the ratio of the average length to the average width of the particles.

The polymer particles are either commercially available or can be produced in a known manner, such as described in U.S. Pat. Nos. 4,489,058 and 4,619,826 and in the International Publications WO 92/00719 and WO 92/00724. Most preferably, the polymers are produced by suspension polymerization.

In one preferred embodiment of the present invention the aqueous hair care composition is a leave-on or rinse-off hair conditioner which additionally comprises a conditioning agent, preferably a cationic conditioning agent. The amount of the conditioning agent preferably is from 0.01 to 10, more preferably from 0.05 to 5, most preferably from 0.1 to 1 percent, based on the total weight of the aqueous hair care composition. Suitable conditioning agents are those which deliver one or more benefits relating to shine, softness, comb-ability, antistatic properties, wet feel, damage, manageability or body of the hair.

Useful conditioning agents are hydrocarbon oils, fatty esters, silicones or, preferably cationic conditioning agents. The aqueous hair care composition may comprise more than one type of conditioning agents.

Preferred silicone-based conditioning agents, generally designated as silicones, are silicone oils, cationic silicones, silicone gums, high refractive silicones, and silicone resins. Suitable silicones are described in the International Patent Application WO 03/047540 A1, pages 16-26, the teaching of which is incorporated herein by reference. Suitable organic conditioning oils, such as hydrocarbon oils, polyolefins, and fatty esters are described on pages 26-29 of WO 03/047540 A1, the teaching of which is incorporated herein by reference. Preferred silicone oils include polyalkyl or polyaryl siloxanes. The silicone atom of each monomeric silicone unit preferably is substituted with two aliphatic or aryl groups which may represent different groups, but preferably they represent the same groups. Preferred alkyl and alkenyl substituents are C₁ to C₅, more preferably C₁ to C₄, most preferably C₁ to C₂ alkyls and alkenyls. Specific non-limiting examples of preferred silicones include: polydimethyl siloxane, polydiethylsiloxane, and polymethylphenylsiloxane. Polydimethylsiloxane is especially preferred.

Preferred cationic conditioning agents are cationic polymers. Suitable cationic polymers are described in the International Patent Application WO 03/047540 A1, pages 10-15, the teaching of which is incorporated herein by reference. Preferred cationic polymers have cationic charge densities of at least 0.4 meq/gm, preferably at least 0.5 meq/gm, but also preferably less than 7 meq/gm, more preferably less than 5 meq/gm, at the pH of intended use of the aqueous hair care composition. The most preferred cationic charge densities are from 0.5 to 2.1 meq/gm. The average molecular weight of such suitable cationic polymers generally is from 10,000 to 10 millions, preferably from 50,000 to 5 millions, more preferably from 100,000 to 3 millions. The “cationic charge density” of a polymer, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain. The cationic polymer preferably contains cationic nitrogen-containing moieties such as quaternary ammonium or cationic protonated amino moieties. The cationic protonated amines can be primary, secondary, or tertiary amines. Known anionic counterions can be use in association with the cationic polymers, preferably halides, such as chloride, fluoride, bromide, or iodide; or sulfate or methylsulfate.

Preferred cationic polymers are quaternary nitrogen-containing polysaccharides, preferably quaternary nitrogen-containing cellulose ethers, such as those described in U.S. Pat. Nos. 3,472,840; 3,962,418; 4,663,159, and U.S. Pat. No. 5,407,919, the teaching of which is incorporated herein by reference. Particularly preferred are quaternary nitrogen-containing hydroxyethyl celluloses. Examples of preferred cationic polymers are salts of hydroxyethyl cellulose reacted with a trimethyl ammonium substituted epoxide, referred to in the industry by the Cosmetic, Toiletry, and Fragrance Association (CTFA) as Polyquaternium-10 and which are commercially available from Amerchol Corp., a subsidiary of The Dow Chemical Company, as UCARE™ Polymer JR-125, UCARE Polymer JR-400, UCARE Polymer KF, UCARE Polymer JR-30M, UCARE Polymer LR-400, UCARE Polymer LR-30M, and UCARE Polymer LK. Examples of other preferred cationic polymers are referred to by CTFA as Polyquaternium-67. They are commercially available from Amerchol Corp. as SoftCAT™ SL 5, SoftCAT SL 30, SoftCAT SL 60, SoftCAT SL 100, SoftCAT SK-L, SoftCAT SK-M, SoftCAT SK-M, SoftCAT SK-MH and SoftCAT SK-H. Other examples of preferred cationic polymers are those referred to in the industry by the CTFA as Polyquaternium-7 with the CAS Registry Number 026590-05-6, and those referred by the CTFA as Polyquaternium-44. Other suitable types of cationic cellulose ethers include the polymeric quaternary ammonium salts of hydroxyethyl cellulose reacted with lauryl dimethyl ammonium- substituted epoxide referred to in the industry (CTFA) as Polyquaternium 24. Other suitable cationic polymers include cationic guar gum derivatives and cationic starch derivatives.

In another aspect of the present invention the aqueous hair care composition is in the form of a shampoo which additionally comprises a surfactant. The aqueous hair care composition may comprise more than one type of surfactants. The total amount of the surfactant is preferably from 5 to 50, more preferably from 10 to 30 percent, based on the total weight of the aqueous hair composition. One type of suitable surfactants are the detersive surfactants described in the International Patent Application WO 03/047540 A1, pages 3-8, the teaching of which is incorporated herein by reference. Anionic, nonionic, zwitterionic, amphoteric surfactants, and mixtures thereof are preferred. Preferred surfactants include mixtures of non-ionic surfactants and anionic surfactants. Useful zwitterionic surfactants are described in U.S. Pat. No. 3,929,678.

Anionic surfactants useful herein are disclosed in U.S. Pat. No. 4,285,841, Barrat et al, issued Aug. 25, 1981, and in U.S. Pat. No. 3,919,678, Laughlin et al, issued Dec. 30, 1975. Preferred anionic surfactants include C₁₁-C₁₈ alkyl benzene sulfonates and primary or branched-chain C₁₀-C₂₀ alkyl sulfates, unsaturated sulfates such as oleyl sulfate, the C₁₀-C₁₈ alkyl alkoxy sulfates, particularly those comprising 1-7 ethoxy groups, C₁₀-C₁₈ alkyl alkoxy carboxylates, particularly those comprising 1-5 ethoxy groups, the C₁₀-C₁₈ glycerol ethers, the C₁₀-C₁₈ alkyl polyglycosides and their corresponding sulfated polyglycosides, and C₁₂-C₁₈ alpha-sulfonated fatty acid esters. Other useful anionic surfactants include water-soluble salts, particularly the alkali metal, ammonium and alkylolammonium salts, such as monoethanolammonium or triethanolammonium salts, of organic sulfuric reaction products having in their molecular structure an alkyl group containing from about 10 to about 20 carbon atoms and a sulfonic acid or sulfuric acid ester group. Other anionic surfactants useful herein are the water-soluble salts of alkyl phenol ethylene oxide ether sulfates and water-soluble salts of esters of alpha-sulfonated fatty acids. The anionic surfactants based on fatty acids include saturated and/or unsaturated fatty acids obtained from natural sources or synthetically prepared. Examples of suitable fatty acids include, but are not limited to, capric, lauric, myristic, palmitic, stearic, arachidic, and behenic acid. Other fatty acids include palmitoleic, oleic, linoleic, linolenic, and ricinoleic acid. Examples of particularly preferred surfactants are fatty acid salts, sulfonates or quaternary ammonium salts, and especially sodium lauryl sulfate (SLS) or sodium laureth sulfate (SLES).

Suitable nonionic surfactants are disclosed in U.S. Pat. No. 3,929,678, Laughlin et al., issued Dec. 30, 1975, and U.S. Pat. No. 4,285,841, Barrat et al, issued Aug. 25, 1981. Exemplary, non- limiting classes of useful nonionic surfactants include C₈-C₁₈ alkyl ethoxylates, with 1-22 ethylene oxide units and C₆-C₁₂ alkyl phenol alkoxylates, particularly ethoxylates and mixed ethoxylates/propoxylates, alkyl dialkyl amine oxides, alkanoyl glucose amides, and mixtures thereof. Other useful nonionic surfactants are polyethylene, polypropylene, and polybutylene oxide condensates of alkyl phenols. These compounds are commonly referred to as alkyl phenol alkoxylates, preferably alkyl phenol ethoxylates. Further useful nonionic surfactants are the condensation products of aliphatic alcohols with from 1 to 25 moles of ethylene oxide. The alkyl chain of the aliphatic alcohol can either be straight or branched, primary or secondary, and generally contains from 8 to 22 carbon atoms. This category of nonionic surfactant is referred to generally as “alkyl ethoxylates.” Other useful nonionic surfactants are the condensation products of ethylene oxide with a hydrophobic base formed by the condensation of propylene oxide with propylene glycol. Further useful surfactants are the condensation products of ethylene oxide with the product resulting from the reaction of propylene oxide and ethylenediamine. Fatty acid amide surfactants, C₁₂-C₁₈ betaines and sulfobetaines (sultaines) are also known surfactants.

An aqueous hair care composition in the form of a shampoo preferably comprises a conditioning agent in addition to the plurality of crosslinked polymer particles and one or more surfactants described above. Useful types and amounts of conditioning agents are described further above.

The aqueous hair care composition preferably comprises one or more viscosity modifiers, more preferably one or more polymeric thickeners as an additional component. The total amount of the viscosity modifier, if present, preferably is from 0.02 to 10, more preferably from 0.5 to 2, most preferably from 0.1 to 1.0 percent, based on the total weight of the composition. Commercially available viscosity modifiers highly useful herein include Carbomers with tradenames Carbopol 934, Carbopol 940, Carbopol 950, Carbopol 980, and Carbopol 981, all available from Noveon, Inc., methylcellulose with trade name BENECEL, hydroxyethyl cellulose with trade name NATROSOL, hydroxypropyl cellulose with trade name KLUCEL, cetyl hydroxyethyl cellulose with trade name POLYSURF 67, all supplied by Hercules, ethylene oxide and/or propylene oxide based polymers with trade names CARBOWAX PEGs, POLYOX WSRs, and UCON FLUIDS, and hydroxyethyl celluloses with the trademark CELLOSIZE, all supplied by Amerchol, and methylcelluloses and hydroxypropyl methylcelluloses with the trademark METHOCEL, all supplied by The Dow Chemical Company.

The aqueous hair care composition of the present invention may further comprise one or more optional components other than the above-described components. Individual concentrations of such optional components typically range from 0.001 to 10 percent by weight of the aqueous hair care compositions. Non-limiting examples of optional components for use in the aqueous hair care composition include anti dandruff agents, suspending agents, dyes, pearlescent aids, foam boosters, pH adjusting agents, perfumes, preservatives, chelants, proteins, and vitamins.

The aqueous hair care composition of the present invention preferably comprises from 10 to 99 percent, more preferably from 50 to 98 percent, most preferably from 80 to 95 percent of water, based on the total weight of the aqueous hair care composition.

The present invention is further illustrated by the following examples which should not be construed to limit the scope of the present invention. All parts and percentages are by weight unless otherwise indicated.

### Example 1 and Comparative Example A

A leave-in-hair conditioner is prepared from the composition of Table 1. The conditioner of Example 1 comprises 5 weight percent of cross-linked polymer particles. The polymer in the cross-linked polymer particles is a copolymer of 56 weight percent of isobornylmethacrylate and 44 weight percent of laurylmethacrylate which is cross-linked with 0.5 weight percent of divinylbenzene. The copolymers are produced by suspension polymerization as described in U.S. Pat. No. 4,619,826 and U.S. Pat. No. 4,489,058.

The conditioner of Comparative Example A comprises an additional amount of 5 weight percent of water instead of the cross-linked polymer particles.

TABLE 1

| | | Comparative |
|---|---|---|
| | Example 1 | Example A |
| Composition of leave-in hair conditioner | (wt. %) | (wt. %) |
| Tego Alkanol 16 (Cetyl Alcohol from Degussa) | 1 | 1 |
| Promulgen G (ceteareth-20 from Noveon) | 1 | 1 |
| Carsoquat SDQ-85 (Ammonium | 1 | 1 |
| benzyldimethyloctadecyl chloride from Lonza) | | |
| PCG-10 (Hydroxyethyl cellulose from Dow) | 25 | 25 |
| (4.0%) | | |
| Glydant (DMDM hydantoin from Lonza) | 0.4 | 0.4 |
| cross-linked polymer particles | 5.0 | — |
| Distilled water | 66.6 | 71.5 |
| Total | 100 | 100 |

Three grams of virgin brown hair is used that is 20 cm long and that has been obtained from International Hair Importers and Products, Inc. Each tress is presoaked for about two minutes with tap water at room temperature and then pre-washed with Tergitol™ 15-S-9 surfactant and rinsed thoroughly. Tergitol 15-S-9 is a mixture of linear secondary alcohols reacted with ethylene oxide of the general formula of C₁₁₋₁₅H₂₃₋₃₁O[CH₂CH₂O]ₓH. Hair tresses are hung vertically and air-dried overnight.

According to Example 1 and Comparative Example A 0.5 g of a leave-in-hair conditioner shown in Table 1 is applied to different hair tresses of 3 g each. 0.25 g of the conditioner is applied to each side of the tress and rubbed in. After allowing sufficient time for drying, artificial sebum (62% Triolein, 11% Squalene and 27% Oleic acid from Aldrich) is applied along the length of the hair tress. The weight of the artificial sebum corresponds to 2, 3 and 4 times the weight of the cross-linked polymer particles in the conditioner of Example 1.

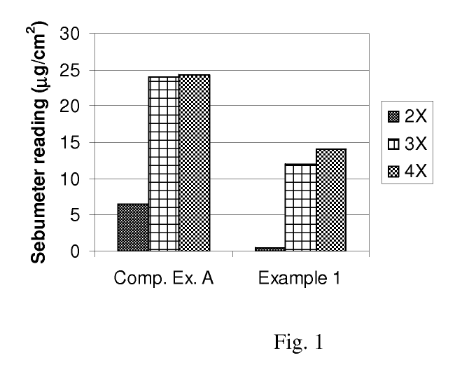

A sebumeter (SM 815® from Courage-Khazaka) is used to measure the oil content at seven different points along the length of the hair tress. Both sides were measured, making a total of 14 readings that were averaged and are shown in FIG. 1. FIG. 1 illustrates that the conditioner of the present invention comprising the cross-linked polymer particles is effective to significantly reduce the amount of sebum from hair tresses.

The hair tresses treated with the leave-in-hair conditioner of Example 1 and of Comparative Example A that have been treated with artificial sebum that corresponds twice the weight of the cross-linked polymer particles in the conditioner of Example 1 are mounted on circular rolls in a shine box apparatus. 10 panelists are asked to determine the oilier tress. All ten panelists determine the tress of Comparative Example A to be shinier and to look oilier.

A panel study is conducted in which 5 panelists are asked to come with their hair washed. An initial visual rating and seven sebumeter readings for each half of the hair is taken. 1 ml of the leave-in hair conditioners of Example 1 and of Comparative Example A are applied to each half of the hair. Visual ratings and seven sebumeter readings are taken at 0.5 hours and 6 hours.

FIG. 2 shows the average of seven sebumeter readings at each time of each of the five panelists with the leave-in hair conditioner of Comparative Example A. Not much change is observed.

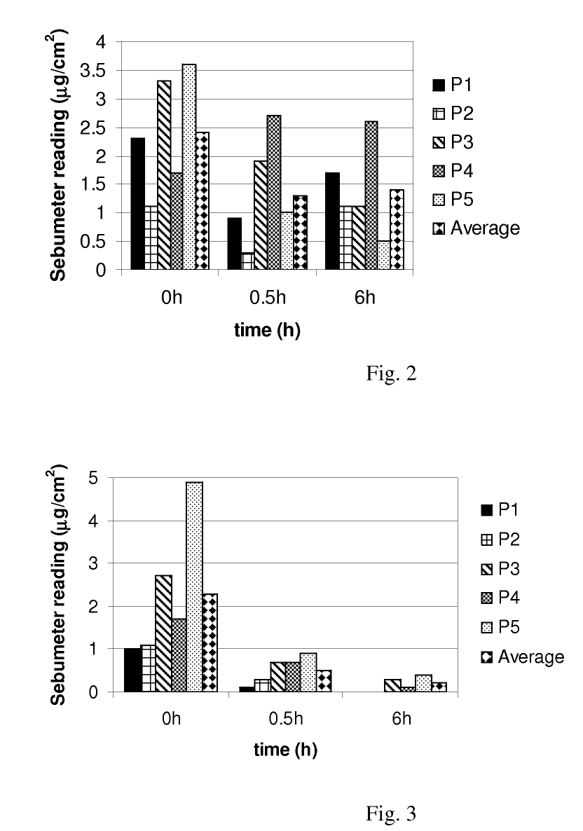

FIG. 3 shows the sebumeter readings of the same panelists with the conditioner of Example 1. It is obvious that the leave-on hair conditioner of Example 1 reduces the sebum on hair.

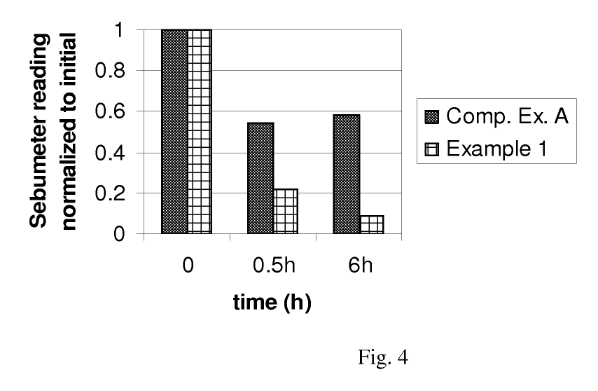

FIG. 4 shows the average sebumeter reading of all panelists normalized to their initial value. It illustrates that the aqueous hair care composition of the present invention is effective in suppressing sebum levels.

### Example 2 and Comparative Examples B and C

1 g of the leave-in-hair conditioner of Example 1 is applied to 3 g of a hair tress described in Example 1.

The leave-in-hair conditioner of Comparative Example B is the same as the conditioner of Example 1, except that it comprises 5 weight percent of Silica Shells, commercially available from Kobo Products, Inc. instead of the cross-linked polymer particles of Example 1.

The leave-in hair conditioner of Comparative Example C is the same as the conditioner of Example 1, except that it comprises 5 weight percent of Polypore E-200, commercially available from Amcol Health and Beauty Solutions instead of the cross-linked polymer particles of Example 1.

Silica Shells and Polypore E-200 are commercially available sebum control ingredients. 1 g of the hair conditioner of Comparative Examples A and B are applied to hair tresses of 3 g each.

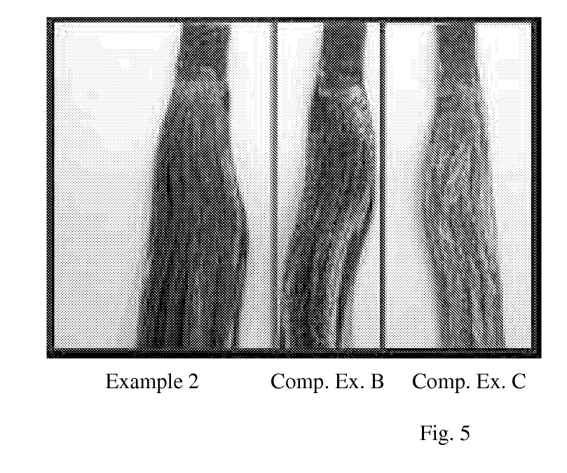

The hair tresses are photographed. FIG. 5 represents the printed photographs. FIG. 5 illustrates that the aqueous hair care composition of the present invention leaves a substantially reduced degree of whiteness on hair, when compared to comparable known aqueous hair care compositions.

1. An aqueous hair care composition comprising a plurality of cross-linked polymer particles of copolymerized isobornylmethacrylate and laurylmethacrylate, wherein the particles are present in an amount sufficient to suppress sebum build up on hair after application of the composition.

2. The hair care composition of claim 1 in the form of a shampoo additionally comprising a surfactant.

3. The hair care composition of claim 1 in the form of a leave-on or rinse-off hair conditioner additionally comprising a conditioning agent.

4.-5. (canceled)

6. The hair care composition of any one of claims 1 wherein the particles have a volume average particle size of from 2 to 30 micrometers at their smallest diameters.

7.-10. (canceled)

11. Use of the hair care composition of any one of claims 1 cleansing or conditioning human hair.

12. Use of the hair care composition of any one of claims 1 for sebum control on hair.

13. A method of suppressing sebum build up on hair between washings, comprising applying to the hair an aqueous hair care composition comprising a plurality of cross-linked polymer particles of copolymerized isobornylmethacrylate and laurylmethacrylate.
